(19) United States
(12) Patent Application Publication
(10) Pub. No.: US 2021/0220086 A1
(43) Pub. Date: Jul. 22, 2021

(54) METHOD AND SYSTEM FOR REMOTE ORTHODONTIC DIAGNOSIS AND TREATMENT PLANNING

(71) Applicant: Daniel S. German, Beachwood, OH (US)

(72) Inventor: Daniel S. German, Beachwood, OH (US)

(21) Appl. No.: 17/156,118

(22) Filed: Jan. 22, 2021

Related U.S. Application Data
(60) Provisional application No. 62/964,169, filed on Jan. 22, 2020.

(51) Int. Cl. A61C 7/00; A61C 9/00; A61C 7/02; G09B 5/06

(52) U.S. Cl. CPC A61C 7/002; G09B 5/065; A61C 7/02; A61C 9/0053

(57) ABSTRACT

A formalized method and system of collaborative care includes providing orthodontic care via collaboration between a dentist, an orthodontic specialist, and a software platform. A method for generating an orthodontic treatment plan includes obtaining patient images, submitting the patient images to an analyzer for diagnostic planning, analyzing the images with the analyzer, generating a treatment objective with the analyzer, calculating time needed for each phase of treatment, determining appliances and instrumentation necessary to achieve the treatment objective, and generating the orthodontic treatment plan. A system for generating an orthodontic treatment plan includes a sensor configured to generate patient images and an analyzer configured to analyze the patient images and generate an orthodontic treatment plan. The system further include a learning management system.

### PRIORITY

This application claims priority to and the benefit of U.S. Provisional Patent Application No. 62/964,169 filed on Jan. 22, 2020.

### TECHNICAL FIELD

The disclosed relates generally to a method and system for providing collaborative orthodontic care, and, more specifically, to a method and system for generating orthodontic treatment plans.

### BACKGROUND

The number of patients in need of orthodontic care surpasses the number of orthodontists capable of providing that care. Approximately 35% of all counties located in the United States have an orthodontic specialist. Further, the ratio of dentists to orthodontists in the United State is approximately 20:1. Many dentists have little to no experience in ordering orthodontic care yet have much of the infrastructure and technical skills to offer that care. While over 65% of the United States population could benefit from orthodontic care, many do not have access due to geographical limitations and a limited number of orthodontic specialists in the country. Further, during auspicious times of widespread illness such as a global pandemic, there is a uniform desire to reduce the amount of in-person human interaction. Thus, there is an unmet demand for orthodontic care.

Accordingly, there is a need for a system of providing orthodontic care to a larger population. More particularly, a collaborative method and system of equipping dentists with the skills and materials necessary to offer orthodontic care in order to meet the current population demands.

### SUMMARY

The following presents a summary of this disclosure to provide a basic understanding of some aspects. This summary is intended to neither identify key or critical elements nor define any limitations of examples or claims. Furthermore, this summary may provide a simplified overview of some aspects that may be described in greater detail in other portions of this disclosure.

Disclosed is a formalized method and system of collaborative care. The method includes providing orthodontic care via collaboration between a dentist, an orthodontic specialist, and a software platform. In one aspect, the method includes obtaining patient images, submitting the images to a collaborative software platform for analysis and strategic planning, reviewing the images, generating a treatment plan, estimating time for each phase of treatment, providing education and training, designing appliances, providing appliances and instrumentation necessary to provide care, reviewing progress of treatment, and providing on-demand support related to treatment plans, office management, or any other aspect necessary to equip a dentist to provide orthodontic care.

### BRIEF DESCRIPTION

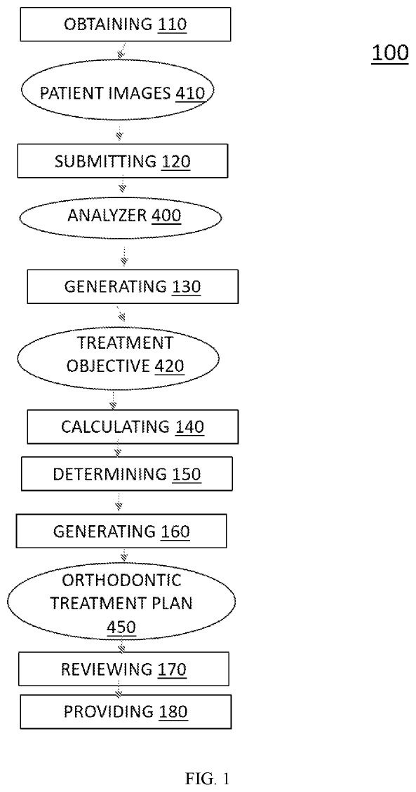

FIG. 1 is a flow chart of a method for generating an orthodontic treatment plan;

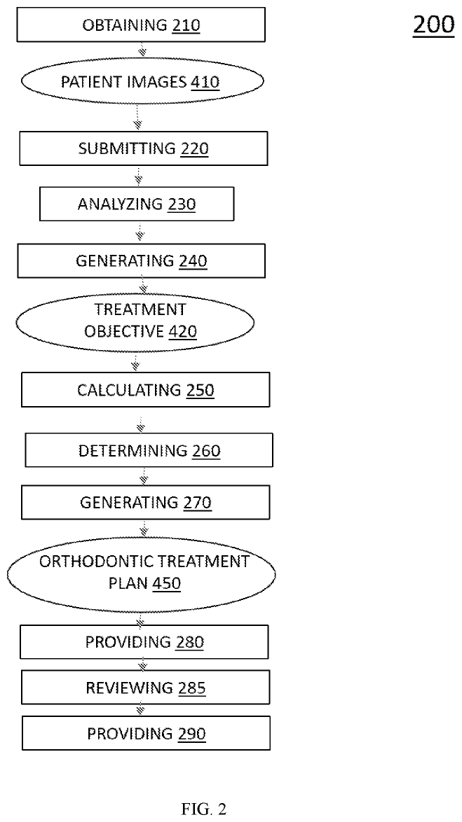

FIG. 2 is a flow chart of a method for delivering remote orthodontic treatment; and

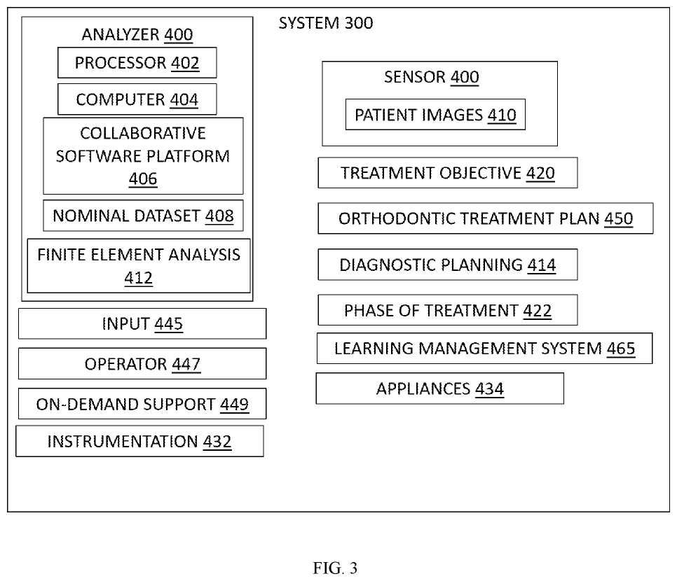

FIG. 3 is a block diagram of a system for generating an orthodontic treatment plan.

### DESCRIPTION

Reference will now be made in detail to exemplary embodiments of the present teachings, examples of which are illustrated in the accompanying drawings. It is to be understood that other embodiments may be utilized and structural and functional changes may be made without departing from the respective scope of the present teachings. Moreover, features of the various embodiments may be combined or altered without departing from the scope of the present teachings. As such, the following description is presented by way of illustration only and should not limit in any way the various alternatives and modifications that may be made to the illustrated embodiments and still be within the spirit and scope of the present teachings.

As used herein, the words “example” and “exemplary” mean an instance, or illustration. The words “example” or “exemplary” do not indicate a key or preferred aspect or example. The word “or” is intended to be inclusive rather an exclusive, unless context suggests otherwise. As an example, the phrase “A employs B or C,” includes any inclusive permutation (e.g., A employs B; A employs C; or A employs both B and C). As another matter, the articles “a” and “an” are generally intended to mean “one or more” unless context suggests otherwise.

It is noted that the various examples described herein may include other components and/or functionality. It is further noted that while various examples refer to braces, various other systems and aligners may be utilized in view of examples described herein. For example, examples may be utilized in traditional braces, aligners, orthopedic jaw modifiers, or other appliances 434 used for orthodontic and dental treatment. As such, references to braces, aligners, orthopedic jaw modifiers, and the like, are understood to include the various other appliances 434.

FIGS. 1 and 2 are flow charts of methods 100 and 200. The methods 100 and 200 achieve the overall goal of providing a means for a dentist to collaborate with an orthodontic specialist through the use of an analyzer 400 to provide orthodontic care. In one or more examples, the dentist begins by selecting a patient to receive orthodontic care. Because not all patients are suitable for this method of providing care, the dentist must consider various factors including severity of malocclusions and medical history. If unsure, the dentist has the option of collaborating with the orthodontic specialist prior to committing to providing orthodontic care. In this instance, the dentist may take intraoral images via a camera, intraoral scanner, x-ray, cone beam imaging, or any other suitable means of obtaining images of the teeth. The dentist will then upload the images to the analyzer 400 comprising a case selection advice software platform 406 that is accessible by the orthodontic specialist. The software platform 406 is configured for analysis of the images, as well as any input 445 from the dentist, to produce a deliverable for the orthodontic specialist to the review. The deliverable includes patient-specific data for providing case selection advice, a five-step patient acquisition process, an estimated time of treatment for various methods of treatment including traditional braces, orthopedic jaw modifiers, or aligners, the likelihood of success with each treatment option, and a recommendation for treatment. The orthodontic specialist may review the deliverable and provide additional information, instruction, or feedback, including quality of the images received.

Once a patient is selected, the dentist may then obtain images of the teeth, if not already completed. The images may then be uploaded to a treatment plan 450 software platform 406 for analysis and strategic planning. The treatment plan 450 software platform 406 is a collaborative, analytic tool for the dentist and orthodontic specialist to utilize in order to achieve the best outcome for the patient.

The treatment plan 450 software platform 406 generates a deliverable, or perfect smile plan (PSP), that outlines various aspects of the overall treatment plan 450. In one aspect, the PSP includes an estimated overall treatment time and a breakdown of treatment time for each 5 stage of the overall plan. In another aspect, the PSP includes clinical findings, a list of identified problems, treatment objective 420s, a prognosis for each objective 420, and step-by-step appointment instructions, including relevant instructional/educational videos and collateral materials for providing treatment. In another aspect, the options for post-orthodontic smile enhancements including appliances 434, teeth whitening, cosmetic bonding, veneers, and the like are generated to tailor the specific needs of the patient and achieve high customer satisfaction. In another aspect, financing options including paid in full, third party financing, or other payments plans are generated to align with the various options presented in the PSP. The dentist may then review these options with the orthodontic specialist and determine the best course of action to present to the patient. Once a plan is selected, the dentist will review the plan with the patient and may use an informed consent as provided by the PSP, including defined outcomes and expectations.

Several collaborative tools are available to the dentist and dental support staff in order to equip them with the necessary skillset to provide the services in the perfect smile plan. In one aspect, the dentist may optionally choose receive additional training if needed to provide the care as outlined in the smile perfect plan. Training may be offered by the orthodontic specialist in various forms. One form includes live, in person seminars with hands-on exercises for the dentist and staff. In another form, training may be achieved via live, remote training tailed to the current patient needs. In another form, the software platform 406 includes on-demand educational videos that the dentist and staff may view as needed. Further, the PSP may include links to training videos specific to each step of the treatment plan 450. This tailored educational series allows for the dentist to develop skills specific to treating that patient and carry those skills forward to treat future patients.

All forms of training and education offered through the collaborative care system may be tracked, monitored, and analyzed in a Learning management system 465 (LMS). The LMS allows for the orthodontic specialist and dentist to collaboratively equip the dentist and dental support staff with the necessary skillset to conduct an orthodontic exam, take orthodontic images, and provide orthodontic care. Further, the LMS may recommend education specific to the dentist and dental support staff based upon past education and input 445 from the dentist. In another aspect, the LMS tracks educational and training history for the purpose to receiving continuing education credit.

Once the dentist is equipped with the necessary skillset to perform the treatment outlined in the PSP, the dentist will review treatment options with the patient and determine the best course of action. If the treatment plan 450 requires the use of appliances 434, the dentist may collaborate with the orthodontic specialist and the software platform 406 for both individualized digital design of appliances 434 and manufacture/delivery of the appliances 434. If the treatment plan 450 calls for use of braces, aligners, expanders, orthopedic jaw modifiers, or other equivalent means, the orthodontic specialist will provide packages including all necessary equipment and instrumentation 432 to provide the planned care. This streamlines the process and eliminates the need for the dentist to create a supply chain to obtain orthodontic appliances 434 and materials.

Once the treatment plan 450 and necessary materials align, the dentist may continue to collaborate with the orthodontic specialist via concierge support for any additional needs that arise. This may include reviewing images of patient progress throughout treatment, in-person or remote education and training, practice management and administrative advice, insurance and scheduling counseling, preparation of marketing materials, and a live chat option (either typed, audio, or video) to provide tailored advice regarding patient care.

In one or more examples, a method 100 for generating an orthodontic treatment plan 450 is disclosed. In an example, the method 100 comprises obtaining 110 patient images 410. The obtaining 110 patient images 410 may be achieved by utilizing a sensor 400 to scan a patient's intraoral cavity. In an example, the obtaining patient images 410 comprises using an optical sensor 400.

In one or more examples, the method 100 comprises submitting 120 the patient images 410 to an analyzer 400 for diagnostic planning 414. In an example, the submitting 120 is achieved by uploading the patient images 410 to a cloud-based storage database accessible to the analyzer 400. In another example, the submitting 120 is achieved by directly uploading the patient images 410 to the analyzer 400.

In one or more examples, the method 100 comprises analyzing the patient images 410 with the analyzer 400. In an example, the analyzer 400 utilizes finite element analysis 412 to compare the patient images 410 to a nominal dataset 408. The nominal dataset 408 may include one or more of a point cloud, plurality of images illustrating dental malocclusions and jaw irregularities, or input 445 manually entered by an operator 447.

In one or more examples, the method 100 comprises generating 130 a treatment objective 420 with the analyzer 400. The analyzer 400 comprises a collaborative software platform 406, a processor 402, and a computer 404. The analyzer 400 is configured to receive input 445 from an operator 447. In an example, the collaborative software platform 406 is configured to receive input 445 from an operator 447. In one example, the operator 447 is an orthodontic specialist. In another example, the operator 447 is a dental practitioner. The analyzer 400 is configured to utilize one or more nominal dataset 408 in conjunction with the patient images 410 and input 445 received from an operator 447 to generate a treatment objective 420.

In another example, the generating 130 a treatment objective 420 comprises comparing analyzed images to a nominal dataset 408 and determining a patient diagnosis. The nominal dataset 408 may include one or more of a point cloud, plurality of images illustrating dental malocclusions and jaw irregularities, or input 445 manually entered by an operator 447.

In one or more examples, the method 100 comprises calculating 140 time needed for each phase of treatment 422. The calculating 140 is achieved by the analyzer 400. In an example, the calculating 140 time for each phase of treatment 422 is based upon a comparison of the treatment objective 420 to a nominal dataset 408. The nominal dataset 408 may include one or more of a point cloud, plurality of images illustrating dental malocclusions and jaw irregularities, or input 445 manually entered by an operator 447. In another example, the nominal dataset 408 includes information on average time needed to fix any one of the dental malocclusions and jaw irregularities.

In one or more examples, the method 100 comprises determining 150 appliances 434 and instrumentation 432 necessary to achieve the treatment objective 420. Appliances 434 include, but are not limited to, traditional braces, aligners, orthopedic jaw modifiers, expanders, or other appliances 434 used for orthodontic and dental treatment. In an example, the method 100 comprises designing appliances 434 tailored to achieving the treatment objective 420. Instrumentation 432 necessary to achieve the treatment objective 420 includes, but is not limited to, pliers, tweezers, wire cutters, tofflemires, scalers, and hemostats.

In one or more examples, the method 100 comprises generating 160 the orthodontic treatment plan 450. The orthodontic treatment plan 450 is a comprehensive outline having at least one phase curated to meet the treatment objective 420. For example, the orthodontic treatment plan 450 includes at least one of an outline having detailed instructions for treating the determined diagnosis, images, milestones, instrumentation 432 needed at each phase of treatment 422, appliances 434 needed at each phase of treatment 422, and any other relevant information for achieving the treatment objective 420.

In one or more examples, the method 100 further comprises reviewing 170 progress of the orthodontic treatment plan 450 and providing 180 on-demand support 449 related to achieving the treatment objective 420. The analyzer 400 may work in conjunction with a learning management system 465 to track progress including milestones that have been met or missed, any deviations from projected milestones, effectiveness of the orthodontic treatment plan 450 with respect to the treatment objective 420, and any other relevant data for achieving the treatment objective 420.

Referring to FIG. 3, in one or more examples, a system 300 for generating an orthodontic treatment plan 450 is disclosed. The system 300 comprises a sensor 400 configured to generate patient images 410. The sensor 400 is configured to scan a patient's intraoral cavity to generate the patient images 410. In an example, the sensor 400 is an optical sensor 400. The sensor 400 is further configured to communicate with an analyzer 400.

In one or more examples, the system 300 comprises an analyzer 400 configured to analyze the patient images 410 and generate an orthodontic treatment plan 450. In an example, the analyzer 400 comprises a collaborative software platform 406, a processor 402, and a computer 404. In an example, the analyzer 400 utilizes finite element analysis 412 to generate the orthodontic treatment plan 450 such that it compares images associated with a treatment objective 420 to those generated by the sensor 400.

The collaborative software platform 406 is configured to receive input 445 from an operator 447. In another example, the analyzer 400 is configured to receive input 445 from an operator 447. In one example, the operator 447 is an orthodontic specialist. In another example, the operator 447 is a dental practitioner.

In one or more examples, the analyzer 400 is configured to generate an orthodontic treatment plan 450 based upon comparison of the patient images 410 to a nominal dataset 408. The nominal dataset 408 may include one or more of a point cloud, plurality of images illustrating dental malocclusions and jaw irregularities, or input 445 manually entered by an operator 447. In another example, the nominal dataset 408 includes information on average time needed to fix any one of the dental malocclusions and jaw irregularities.

In one or more examples, the analyzer 400 is further configured to design appliances 434 tailored to achieving a treatment objective 420. In another example, the analyzer 400 is further configured to select instrumentation 432 tailored to achieving a treatment objective 420. Appliances 434 include, but are not limited to, traditional braces, aligners, orthopedic jaw modifiers, expanders, or other appliances 434 used for orthodontic and dental treatment. In an example, the method 100 comprises designing 190 appliances 434 tailored to achieving the treatment objective 420. Instrumentation 432 necessary to achieve the treatment objective 420 includes, but is not limited to, pliers, tweezers, wire cutters, tofflemires, scalers, and hemostats.

In one or more examples, the system 300 further comprises a learning management system 465. The analyzer 400 may work in conjunction with a learning management system 465 to track progress including milestones that have been met or missed, any deviations from projected milestones, effectiveness of the orthodontic treatment plan 450 with respect to the treatment objective 420, and any other relevant data for achieving the treatment objective 420.

In one or more examples, a method 200 for delivering remote orthodontic treatment is disclosed. The method 200 comprises obtaining 210 patient images 410. The obtaining 210 patient images 410 may be achieved by utilizing a sensor 400 to scan a patient's intraoral cavity. In an example, the obtaining patient images 410 comprises using an optical sensor 400.

In one or more examples, the method 200 comprises submitting 220 the patient images 410 to an analyzer 400 for diagnostic planning 414. In an example, the submitting 220 is achieved by uploading the patient images 410 410 to a cloud-based storage database accessible to the analyzer 400. In another example, the submitting 220 is achieved by directly uploading the patient images 410 to the analyzer 400.

In one or more examples, the method 200 comprises analyzing 230 the patient images 410 with the analyzer 400. In an example, the analyzer 400 utilizes finite element analysis 412 to compare the patient images 410 to a nominal dataset 408. The nominal dataset 408 may include one or more of a point cloud, plurality of images illustrating dental malocclusions and jaw irregularities, or input 445 manually entered by an operator 447.

In one or more examples, the method 200 comprises generating 240 a treatment objective 420 with the analyzer 400. The analyzer 400 comprises a collaborative software platform 406, a processor 402, and a computer 404. The analyzer 400 is configured to receive input 445 from an operator 447. In an example, the collaborative software platform 406 is configured to receive input 445 from an operator 447. In one example, the operator 447 is an orthodontic specialist. In another example, the operator 447 is a dental practitioner. The analyzer 400 is configured to utilize one or more nominal dataset 408 in conjunction with the patient images 410 and input 445 received from an operator 447 to generate a treatment objective 420.

In another example, the generating 240 a treatment objective 420 comprises comparing analyzed images to a nominal dataset 408 and determining a patient diagnosis. The nominal dataset 408 may include one or more of a point cloud, plurality of images illustrating dental malocclusions and jaw irregularities, or input 445 manually entered by an operator 447.

In one or more examples, the method 200 comprises calculating 250 time needed for each phase of treatment 422. The calculating 250 is achieved by the analyzer 400. In an example, the calculating 160 time for each phase of treatment 422 is based upon a comparison of the treatment objective 420 to a nominal dataset 408. The nominal dataset 408 may include one or more of a point cloud, plurality of images illustrating dental malocclusions and jaw irregularities, or input 445 manually entered by an operator 447. In another example, the nominal dataset 408 includes information on average time needed to fix any one of the dental malocclusions and jaw irregularities.

In one or more examples, the method 200 comprises determining 260 appliances 434 and instrumentation 432 necessary to achieve the treatment objective 420. Appliances 434 include, but are not limited to, traditional braces, aligners, orthopedic jaw modifiers, expanders, or other appliances 434 used for orthodontic and dental treatment. In an example, the method 100 comprises designing 190 appliances 434 tailored to achieving the treatment objective 420. Instrumentation 432 necessary to achieve the treatment objective 420 includes, but is not limited to, pliers, tweezers, wire cutters, tofflemires, scalers, and hemostats.

In one or more examples, the method 200 comprises generating 270 the orthodontic treatment plan 450. The orthodontic treatment plan 450 is a comprehensive outline having at least one phase curated to meet the treatment objective 420. For example, the orthodontic treatment plan 450 includes at least one of an outline having detailed instructions for treating the determined diagnosis, images, milestones, instrumentation 432 needed at each phase of treatment 422, appliances 434 needed at each phase of treatment 422, and any other relevant information for achieving the treatment objective 420.

In one or more examples, the method 200 comprises providing 280 education and training to a practitioner. Providing 280 education and training to a practitioner may be achieved through a learning management system 465. The analyzer 400 may work in conjunction with the learning management system 465 to track progress including milestones that have been met or missed, any deviations from projected milestones, effectiveness of the orthodontic treatment plan 450 with respect to the treatment objective 420, and any other relevant data for achieving the treatment objective 420.

In one or more examples, the method 200 comprises reviewing 285 progress of the orthodontic treatment plan 450. Reviewing progress of the orthodontic treatment plan 450 may be achieved through the collaborative software platform 406 of the analyzer 400. The collaborative software platform 406 may utilize one or more of artificial intelligence, input 445 from a user, a nominal dataset 408, or any other information relevant to reviewing progress related to achieving the treatment objective 420 and successful completion of the orthodontic treatment plan 450.

In one or more examples, the method 200 comprises providing 290 on-demand support 449 related to the orthodontic treatment plan 450. Providing 290 on-demand support 449 related to the orthodontic treatment plan 450 may be achieved through the collaborative software platform 406 of the analyzer 400. The collaborative software platform 406 may utilize one or more of artificial intelligence, input 445 from a user, a nominal dataset 408, or any other information relevant to offering tailored, on-demand support 449 related to achieving the treatment objective 420 and successful completion of the orthodontic treatment plan 450.

What has been described above includes examples of the present specification. It is, of course, not possible to describe every conceivable combination of components or methodologies for purposes of describing the present specification, but one of ordinary skill in the art may recognize that many further combinations and permutations of the present specification are possible. Each of the methods and systems described above may be combined or added together in any permutation to define examples disclosed herein. Accordingly, the present specification is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A method for generating an orthodontic treatment plan, the method comprising:
obtaining patient images;
submitting the patient images to an analyzer for diagnostic planning;
analyzing the images with the analyzer;
generating a treatment objective with the analyzer;
calculating time needed for each phase of treatment;
determining appliances and instrumentation necessary to achieve the treatment objective; and
generating the orthodontic treatment plan.

2. The method of claim 1 further comprising:
designing appliances tailored to achieving the treatment objective.

3. The method of claim 1, wherein analyzing comprises finite element analysis.

4. The method of claim 1, wherein the calculating time for each phase of treatment is based upon a comparison of the treatment objective to a nominal dataset.

5. The method of claim 1 further comprising:
reviewing progress of the orthodontic treatment plan; and
providing on-demand support related to achieving the treatment objective.

6. The method of claim 1, where the analyzer comprises:
a collaborative software platform;
a processor; and
a computer.

7. The method of claim 6, wherein the collaborative software program is configured to receive input from an operator.

8. The method of claim 1, wherein the obtaining patient images comprises an optical sensor.

9. The method of claim 1, wherein the generating a treatment objective comprises:
comparing analyzed images to a nominal dataset; and
determining a patient diagnosis.

10. An orthodontic treatment plan generated by the method of claim 1.

11. A system for generating an orthodontic treatment plan comprising:
a sensor configured to generate patient images; and
an analyzer configured to analyze the patient images and generate an orthodontic treatment plan.

12. The system of claim 11, wherein the sensor is an optical sensor.

13. The system of claim 11, wherein the analyzer comprises:
a collaborative software program;
a processor; and
a computer.

14. The system of claim 11 wherein the analyzer generates an orthodontic treatment plan based upon comparison of the patient images to a nominal dataset.

15. The system of claim 13, wherein the collaborative software program is configured to receive input from an operator.

16. The system of claim 11, wherein the analyzer utilizes finite element analysis to generate the orthodontic treatment plan.

17. The system of claim 11, wherein the analyzer is further configured to design appliances tailored to achieving a treatment objective.

18. The system of claim 11, wherein the analyzer is further configured to select instrumentation tailored to achieving a treatment objective.

19. The system of claim 11 further comprising a learning management system.

20. A method for delivering remote orthodontic treatment, the method comprising:
obtaining patient images;
submitting the patient images to an analyzer for diagnostic planning;
analyzing the images with the analyzer;
generating a treatment objective with the analyzer;
calculating time needed for each phase of treatment;
determining appliances and instrumentation necessary to achieve the treatment objective;
generating the orthodontic treatment plan;
providing education and training to a practitioner;
reviewing progress of the orthodontic treatment plan; and
providing on-demand support related to the orthodontic treatment plan.
